(19) United States
(12) Patent Application Publication
(10) Pub. No.: US 2009/0029340 A1
(43) Pub. Date: Jan. 29, 2009

(54) Cryoprotective Compositions and Methods of Using Same

(75) Inventor: Eran Gabbai, Kfar-MaAs (IL)

Correspondence Address: FOLEY AND LARDNER LLP;SUITE 500, 3000 K STREET NW, WASHINGTON, DC, 20007, US

(73) Assignee: Do-Coop Technologies Ltd.

(21) Appl. No.: 12/087,429

(22) PCT Filed: Jan. 4, 2007
(86) PCT No.: PCT/IL2007/000013

§ 371 Date: Jul. 3, 2008

Related U.S. Application Data
(60) Provisional application No. 60/755,850, filed on Jan. 4, 2006.
(60) Provisional application No. 60/755,852, filed on Jan. 4, 2006.
(60) Provisional application No. 60/755,851, filed on Jan. 4, 2006.

(30) Foreign Application Priority Data
Jan. 4, 2006 (US) 11/324586

(51) Int. Cl. A01N 1/02; C12N 5/06; A01N 1/00

(52) U.S. Cl. 435/1.3; 435/325; 435/374

(57) ABSTRACT

A cryoprotective composition which comprises nanostructures, liquid and at least one cryoprotective agent is provided.

### FIELD AND BACKGROUND OF THE INVENTION

The present invention relates to a novel cryoprotective composition and methods of using same.

Nature dictates that biological material will decay and die. Whereas refrigeration technology provides a means of slowing the rate of deterioration of perishable goods, the use of much lower temperatures has proved a means of storing living organisms in a state of suspended animation for extended periods. The scientific field of cryobiology formally began following the initial discovery over 50 years ago when live spermatozoa were preserved over long periods of time at sub-zero temperatures using glycerol as an effective protectant [Polge C, Smith A U and Parkes A S (1949), Nature, 164, 666]. This paved the way for the discovery of improved techniques, since if not properly controlled, cryopreservation can lead to cell damage and a decrease in cell viability.

Cryobiology embraces a wide range of applications and has the potential to provide solutions for the long term storage of many types of biological material.

Cell and tissue transplantation is fast becoming an important treatment for several diseases and conditions including, but not limited to, diabetes [Janjic et al., Pancreas 13: 166-172, 1996], heart valve replacement [Feng et al., Eur J Cardiothorac Surg 6: 251-255, 1992], cataracts [Taylor Cryobiology 23: 323-353, 1986], skin replacement [De Luca et al., Burns 15: 303-309, 1989], and plastic and reconstruction surgery [Hibino et al., J Craniomaxillofac Surg 24: 346-351, 1996].

As cell and tissue transplantation gain wider acceptance and use, the need for improved methods for their long term storage also increases.

Cell cryopreservation is particularly relevant to the field of in-vitro fertilization, both for the healthy and non-healthy individual. For example, healthy men may want to donate sperm, especially those exposed to occupational hazards (e.g. irradiation), which must then be preserved. Men undergoing chemotherapy, irradiation or a testicular biopsy may also want to store their sperm prior to treatment in order to retain their fertility.

It is estimated that 20% of the world's population suffer from sub-fertility, 60% of whom are male. In the last fifty years, both the average sperm number and sperm quality has been declining steadily (World Health Organization, 2005). Preservation of sperm from sub-fertile males with very low sperm production (severe oligoteratoasthenozoospermia, O.T.A.) at an early age would increase the chances for these men to have children.

Preservation of sperm from domestic animals, such as bulls and boars aids in their genetic improvement contributing to the milk and meats market.

Preservation of the female reproductive cell and the formation of donor “egg banks” would facilitate and lessen the cost of oocyte donation for women that are unable to produce their own oocytes. Provision of viable storage methods of eggs would benefit women wishing to delay their reproductive choices. Additionally, preservation of ovarian tissue would benefit women about to undergo therapy which may threaten their reproductive health.

Methods for embryo cryopreservation are well established and are routinely used for preserving embryos of women undergoing in-vitro fertilization (IVF). This prevents potential damaging side-effects of continuous hormone treatments in order to stimulate the ovaries each time a woman might wish to produce another child. In addition, IVF treatment is stressful and costly. Cryopreservation helps reduce the inconvenience, discomfort and cost of IVF by reducing the number of egg retrievals a woman must undergo, while offering multiple chances to become pregnant. However, the techniques used are still associated with high technological complexity and a high proportion of frozen embryos do not survive following the thawing procedure (50-60%).

Sperm, egg and embryo preservation is also relevant for the perpetuation of endangered animal species and for the maintenance of founder transgenic animals.

Long-term preservation of entire organs is a particular challenge to the science of cryobiology. Most organ transplantation is performed immediately following the death of the donor. The time that the organ remains ex vivo is minimized so as to reduce anoxic and ischemic damage. The transplanted organ must then function immediately after the recipient is removed from life-support systems with no time for organ recovery or repair. There is also no time (between donor harvest and transplantation) in which to do tissue typing and cross-matching, despite the significant improvement that such measures would confer on the process.

Preservation of the organ following removal, for a sufficient amount of time, would help to overcome many of these problems. Banking of organs would also aid in solving the greatest problem in transplantation medicine, which is the shortfall in organ availability in relation to the total number of transplants that are needed.

However, the process of freezing cells can be harsh as a result of thermal, osmotic, and/or mechanical shock to the cell, and the formation of crystals, which can damage cellular structures, particularly the plasma membrane. In addition, the process of freezing and thawing causes dehydration of the cell with potential for cellular damage. The use of cryoprotectants helps to alleviate some of these problems. Commonly used cryoprotectants include glycerol, hydroxyethyl starch (HES) ethylene glycol and DMSO. Nevertheless, the process of cryopreservation remains encumbered with a low cell viability record and many tissue types and organs are damaged and poorly functioning.

For example, the most acceptable cryoprotective agent for semen is Ackerman's medium which consists of TRIS buffer, egg yolk and glycerol (TES buffer). However glycerol is known to have a toxic effect on sperm survival and function. Thus, although practiced routinely, the sperm cryopreservation technique is associated with only 25-30% cell survivability following the freeze thaw procedure. Fewer are able to fertilize ova and even less lead to vital embryos following cryopreservation [Thomas C A et al., 1998, Bio. Reprod., 58:786-793].

The ability to cryopreserve mammalian oocytes in an easily reproducible manner has not yet been achieved and successes have been sporadic. Persistent concerns have arisen questioning whether freezing and thawing of mature oocytes may disrupt the meiotic spindle and thus increase the potential for aneuploidy in the embryos arising from such eggs. With respect to cryostorage of donated oocytes there have been several reports that have shown some success with this approach (Polak de Fried et al, 1998; Tucker et al, 1998a; Yang et al, 1998). Six pregnancies have generated 10 babies from cryopreserved donor oocytes in these reports. Additionally, use of frozen donor oocytes for ooplasmic transfer has been reported with a successful delivery of a twin following thawed ooplasmic donation (Lanzendorf et al., 1999). Studies cryopreserving mouse oocytes report very different survival and fertilization rates [Carroll et al., 1993; Carroll et al., 1990; Cohen et al., 1988; George et al., 1994; Glenister et al., 1990; Gook et al., 1993; Whittingham et al., 1977].

Although some plant tissues, microalgae and protozoa have been successfully cryopreserved for conservation purposes; many species are unable to undergo successful cryopreservation [Methods in Molecular biology, 14, Cryopreservation and Freeze-Drying Protocols, Humana Press, 1995].

The problems associated with cryopreservation of cells are only exacerbated in the case of tissue cryopreservation and even more so with whole organ cryopreservation. The presence of many different cell types, each with its own requirements for optimal cryopreservation limits the recovery of each when a single thermal protocol is imposed on all of the cells. Extracellular ice can cause mechanical damage to the structural integrity of the tissue or organ, particularly the vascular component, where ice is likely to form. Mechanical fractures occur in the vitreous solids that exist between ice crystals when thermal stresses occur at low temperatures. These fractures separate parts of the organ from each other. There are disruptions of the attachments that form between cells and between cells and their basement membranes. There are mechanical stresses caused by the osmotic movement of interstitial water. Each of these is an additional, and formidable, source of damage.

There is thus a widely recognized need for, and it would be highly advantageous to have, methods and compositions for improving cryoprotection techniques devoid of the above limitations.

### SUMMARY OF THE INVENTION

According to one aspect of the present invention there is provided a cryoprotective composition comprising nanostructures, liquid and at least one cryoprotective agent.

According to another aspect of the present invention there is provided a method of cryopreserving cellular matter comprising contacting the cellular matter with a composition comprising nanostructures and a liquid; and subjecting the cellular matter to a cryopreserving temperature, thereby cryopreserving the cellular matter.

According to yet another aspect of the present invention there is provided a method of recovering cryopreserved cellular matter comprising cryopreserving cellular matter by contacting the cellular matter with a composition comprising nanostructures and a liquid and subjecting the cellular matter to a cryopreserving temperature; thawing the cryoprotected cellular matter; and removing the composition, thereby recovering cryopreserved cellular matter.

According to still another aspect of the present invention there is provided a cryopreservation container comprising the cryoprotective composition comprising nanostructures, liquid and at least one cryoprotective agent.

According to an additional aspect of the present invention there is provided a cryopreservation container comprising nanostructures and a liquid.

According to further features in preferred embodiments of the invention described below, the cryoprotective composition further comprises at least one cryoprotective agent.

According to still further features in the described preferred embodiments, the nanostructures comprise a core material of a nanometric size enveloped by ordered fluid molecules of the liquid, the core material and the envelope of ordered fluid molecules being in a steady physical state.

According to still further features in the described preferred embodiments, the nanostructures are formulated from hydroxyapatite.

According to still further features in the described preferred embodiments, the fluid molecules comprise a heterogeneous fluid composition comprising at least two homogeneous fluid compositions and whereas the liquid is identical to at least one of the at least two homogeneous fluid compositions.

According to still further features in the described preferred embodiments, at least a portion of the fluid molecules are in a gaseous state.

According to still further features in the described preferred embodiments, a concentration of the nanostructures is less than 10²⁰ per liter.

According to still further features in the described preferred embodiments, a concentration of the nanostructures is less than 10¹⁵ per liter.

According to still further features in the described preferred embodiments, the nanostructures are capable of forming clusters.

According to still further features in the described preferred embodiments, the nanostructures are capable of maintaining long range interaction thereamongst.

According to still further features in the described preferred embodiments, the composition is characterized by an enhanced ultrasonic velocity relative to water.

According to still further features in the described preferred embodiments, the According to still further features in the described preferred embodiments, the core material is selected from the group consisting of a ferroelectric material, a ferromagnetic material and a piezoelectric material.

According to still further features in the described preferred embodiments, the core material is a crystalline core material.

According to still further features in the described preferred embodiments, the liquid is water.

According to still further features in the described preferred embodiments, each of the nanostructures is characterized by a specific gravity lower than or equal to a specific gravity of the liquid.

According to still further features in the described preferred embodiments, the nanostructures and liquid comprise a buffering capacity greater than a buffering capacity of water.

According to still further features in the described preferred embodiments, the cryoprotective composition comprises less than 10% by volume glycerol.

According to still further features in the described preferred embodiments, the cryoprotective composition is devoid of glycerol.

According to still further features in the described preferred embodiments, the at least one cryoprotective agent is selected from the group consisting of acetamide, agarose, alginate, 1-analine, albumin, ammonium acetate, butanediol, chondroitin sulfate, chloroform, choline, dextrans, diethylene glycol, dimethyl acetamide, dimethyl formamide, dimethyl sulfoxide (DMSO), erythritol, ethanol, ethylene glycol, formamide, glucose, glycerol, alpha-glycerophosphate, glycerol monoacetate, glycine, hydroxyethyl starch, inositol, lactose, magnesium chloride, magnesium sulfate, maltose, mannitol, mannose, methanol, methyl acetamide, methylformamide, methyl ureas, phenol, pluronic polyols, polyethylene glycol, polyvinylpyrrolidone, proline, propylene glycol, pyridine N-oxide, ribose, serine, sodium bromide, sodium chloride, sodium iodide, sodium nitrate, sodium sulfate, sorbitol, sucrose, trehalose, triethylene glycol, trimethylamine acetate, urea, valine and xylose.

According to still further features in the described preferred embodiments, the cryoprotective composition further comprises a stabilizer.

According to still further features in the described preferred embodiments, the stabilizer is a divalent cation, a radical scavenger, an anti-oxidant, an ethylene inhibitor or a heat-shock protein.

According to still further features in the described preferred embodiments, the ethylene inhibitor is an ethylene biosynthesis inhibitor or an ethylene action inhibitor.

According to still further features in the described preferred embodiments, the cryoprotective composition further comprises a buffer or medium.

According to still further features in the described preferred embodiments, the buffer is a Tris buffer or a phosphate buffer.

According to still further features in the described preferred embodiments, the cellular matter is selected from the group comprising a body fluid, a cell culture, a cell suspension, a cell matrix, a tissue, an organ and an organism.

According to still further features in the described preferred embodiments, the body fluid is semen.

According to still further features in the described preferred embodiments, the semen is derived from an oligospermic, teratospermic or asthenozoospermic male.

According to still further features in the described preferred embodiments, the cellular matter is plant cellular matter.

According to still further features in the described preferred embodiments, the plant matter is selected from the group consisting of a growth needle, a leaf, a root, a bark, a stem, a rhizome, a callus cell, a protoplast, a cell suspension, an organ, a meristem, a seed and an embryo.

According to still further features in the described preferred embodiments, the cellular matter is microorganism cellular matter.

According to still further features in the described preferred embodiments, the cellular matter is mammalian cellular matter.

According to still further features in the described preferred embodiments, the mammalian cellular matter is selected from the group consisting of a stem cell, a sperm, an egg and an embryo.

According to still further features in the described preferred embodiments, the cellular matter is genetically modified.

According to still further features in the described preferred embodiments, the method of cryopreserving cellular matter, further comprises conditioning the cellular matter prior to step (a).

According to still further features in the described preferred embodiments, the conditioning is affected by stabilizer treating, cold acclimatizing, heat-shock treating and/or lyophilizing.

According to still further features in the described preferred embodiments, step (a) and step (b) are performed simultaneously.

According to still further features in the described preferred embodiments, the cryopreserving temperature is less than about −80° C.

The present invention successfully addresses the shortcomings of the presently known configurations by providing novel cryoprotective compositions and methods of cryopreservation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D are bar graphs illustrating the cryoprotective effects of the liquid comprising nanostructures when added to the standard cryoprotection buffer TES. FIG. 1A illustrates the influence of cryopreservation in the presence of liquid comprising nanostructures on sperm vitality. FIG. 1B illustrates the influence of cryopreservation in the presence of liquid comprising nanostructures on sperm motility. FIG. 1C illustrates the influence of cryopreservation in the presence of a liquid comprising nanostructures on sperm fertilization capability. FIG. 1D illustrates the influence of cryopreservation in the presence of liquid comprising nanostructures on sperm DNA fragmentation.

FIG. 2 shows results of isothermal measurement of absolute ultrasonic velocity in the liquid composition of the present invention as a function of observation time.

FIG. 3 is a graph illustrating Sodium hydroxide titration of various water compositions as measured by absorbence at 557 nm.

FIGS. 4A-C are graphs of an experiment performed in triplicate illustrating Sodium hydroxide titration of water comprising nanostructures and RO water as measured by pH.

FIGS. 5A-C are graphs illustrating Sodium hydroxide titration of water comprising nanostructures and RO water as measured by pH, each graph summarizing 3 triplicate experiments.

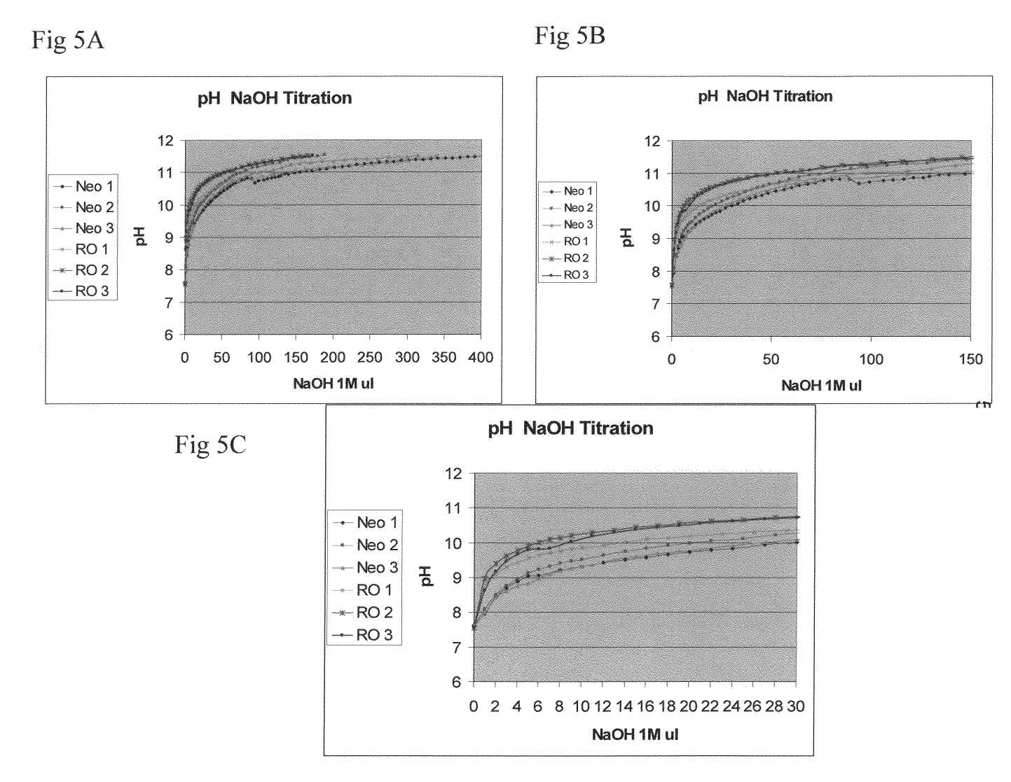

FIGS. 6A-C are graphs of an experiment performed in triplicate illustrating Hydrochloric acid titration of water comprising nanostructures and RO water as measured by pH.

FIG. 7 is a graph illustrating Hydrochloric acid titration of water comprising nanostructures and RO water as measured by pH, the graph summarizing 3 triplicate experiments.

FIGS. 8A-C are graphs illustrating Hydrochloric acid (FIG. 8A) and Sodium hydroxide (FIGS. 8B-C) titration of water comprising nanostructures and RO water as measured by absorbence at 557 nm.

FIGS. 9A-B are photographs of cuvettes following Hydrochloric acid titration of RO (FIG. 9A) and water comprising nanostructures (FIG. 9B). Each cuvette illustrated addition of 1 μl of Hydrochloric acid.

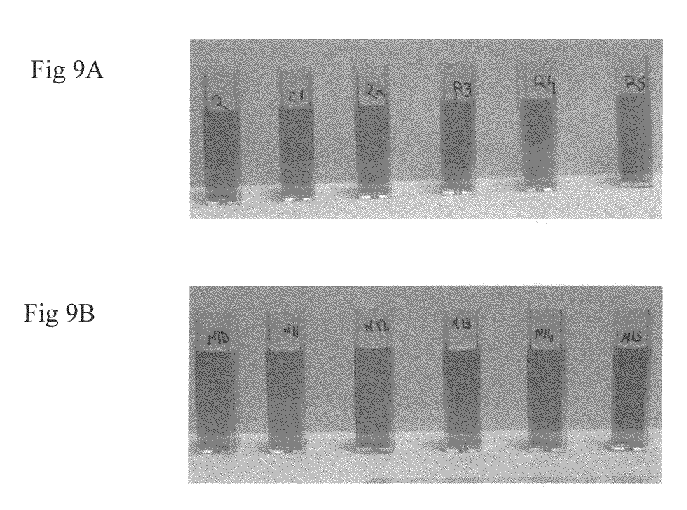

FIGS. 10A-C are graphs illustrating Hydrochloric acid titration of RF water (FIG. 10A), RF2 water (FIG. 10B) and RO water (FIG. 10C). The arrows point to the second radiation.

FIG. 11 is a graph illustrating Hydrochloric acid titration of FR2 water as compared to RO water. The experiment was repeated three times. An average value for all three experiments was plotted for RO water.

FIGS. 12A-B are photographs of a DNA gel stained with ethidium bromide illustrating the PCR products obtained in the presence and absence of the liquid composition comprising nanostructures following heating according to the protocol described in Example 7 using two different Taq polymerases.

FIG. 13 is a photograph of a DNA gel stained with ethidium bromide illustrating the PCR products obtained in the presence and absence of the liquid composition comprising nanostructures following heating according to the protocol described in Example 8 using two different Taq polymerases.

### DESCRIPTION OF THE PREFERRED EMBODIMENTS

The present invention is of a novel cryoprotective composition and methods of using same.

Specifically, the present invention can be used to cryopreserve cellular matter thereby facilitating its storage, transporting and handling.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Cryobiology embraces a wide range of applications and has the potential to provide solutions for the long term storage of many types of biological material. If not properly controlled, however, cryopreservation can lead to cell damage and a decrease in cell viability due to thermal, osmotic, and/or mechanical shock and the formation of crystals, which can damage cellular structures, particularly the plasma membrane. In addition, the process of freezing and thawing causes dehydration of the cell with potential for cellular damage. The use of cryoprotectants (i.e., cryoprotective agents) helps to alleviate some of these problems. Commonly used cryoprotectants include glycerol, hydroxyethyl starch (HES) ethylene glycol and DMSO. Although essential for reducing the injury of cells during freezing and thawing, these cryoprotectants are also toxic to the cell. For example, the toxic effects of glycerol on sperm cells have been reported even at concentrations of less than 2% (Tulandi and McInnes, 1984). Additionally it has been shown that sperm motility decreases as glycerol concentration increases (Weidel and Prins, 1987, J Androl., January-February; 8(1):41-7; Critser et al., 1988, Fertil Steril. August; 50(2):314-20). Furthermore, the presence of cryoprotective agents was shown to provoke sperm-cell injury due to osmotic stress (Critser et al., 1988, Fertil Steril. August; 50(2):314-20).

Therefore, it would be highly advantageous to have novel cryoprotective compositions which are devoid of the above limitations.

While reducing the present invention to practice, the present inventor has uncovered that compositions comprising nanostructures (such as those described in U.S. Pat. Appl. Nos. 60/545,955 and Ser. No. 10/865,955, and International Patent Application, Publication No. WO2005/079153) can be used to efficiently cryoprotect cellular matter.

As illustrated hereinbelow and in the Examples section which follows the present inventor has demonstrated that nanostructures and liquid in the presence of a buffer comprising a cryoprotective agent (glycerol) is more effective than the buffer alone at both protecting sperm cells following cryoprotection and at increasing sperm quality following thawing. The compositions of the present invention may therefore be used to reduce the amount of toxic cryoprotective agents (such as glycerol) necessary for cryoprotection, thereby limiting the cryoprotective agents' deleterious effects.

Thus, according to one aspect of the present invention there is provided a cryoprotective composition comprising nanostructures, liquid and optionally at least one cryoprotective agent.

As used herein the phrase “cryoprotective composition” refers to a liquid composition that reduces the injury of cells (e.g., mechanical injury caused by intracellular and extracellular ice crystal formation; and injury caused by osmotic forces created by changing solute conditions caused by extracellular ice formation) during freezing and thawing.

As used herein, the phrase “cryoprotective agent” refers to a chemical or a chemical solution which facilitates the process of cryoprotection by reducing the injury of cells during freezing and thawing. Preferably, the cryoprotective agent is non-toxic to the cellular matter under the conditions at which it is used (i.e. at a particular concentration, for a particular exposure time and to cells in a medium of a particular osmolarity). According to this aspect of the present invention a cryoprotective agent may be cell permeating or non-permeating. Examples of cryoprotective agents include but are not limited to, dehydrating agents, osmotic agents and vitrification solutes (i.e., solutes that aid in the transformation of a solution to a glass rather than a crystalline solid when exposed to low temperatures).

Without being bound to theory, it is believed that non-permeating cryoprotective agents inhibit the efflux of intracellular water thereby preventing cell shrinkage beyond its minimum critical volume. By reducing cellular retraction, cryoprotective agents attenuate hyperconcentration of the intracellular fluid thereby inhibiting the precipitation of proteins. Permeating cryoprotective agents reduce the amount of ice formed therein, hence reducing the amount of physical injury to cell membranes and organelles.

Preferably, the cryoprotective agent and its concentration are selected on an empirical basis, since each cell responds to an individual cryoprotective agent in a particular way according to its type and environment. Typically, a tissue requires a more penetrating cryoprotective agent than a cell suspension. Conversely, cryoprotection of small cells may not require agents that penetrate cell membranes. In addition, the cryoprotective agent and its concentration are selected according to the method and stage of cryoprotection as further described hereinbelow.

Examples of cryoprotective agents that can be used according to this aspect of the present invention include, but are not limited to acetamide, agarose, alginate, 1-analine, albumin, ammonium acetate, butanediol, chondroitin sulfate, chloroform, choline, dextrans, diethylene glycol, dimethyl acetamide, dimethyl formamide, dimethyl sulfoxide (DMSO), erythritol, ethanol, ethylene glycol, formamide, glucose, glycerol, alpha.-glycerophosphate, glycerol monoacetate, glycine, hydroxyethyl starch, inositol, lactose, magnesium chloride, magnesium sulfate, maltose, mannitol, mannose, methanol, methyl acetamide, methylformamide, methyl ureas, phenol, pluronic polyols, polyethylene glycol, polyvinylpyrrolidone, proline, propylene glycol, pyridine N-oxide, ribose, serine, sodium bromide, sodium chloride, sodium iodide, sodium nitrate, sodium sulfate, sorbitol, sucrose, trehalose, triethylene glycol, trimethylamine acetate, urea, valine and xylose.

Preferably the cryoprotective composition of the present invention comprises less than 20% glycerol and even more preferably is devoid of glycerol (for the reasons described hereinabove).

As mentioned the cryoprotective compositions of this aspect of the present invention further comprise nanostructures and liquid.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, in which case the nanostructure is referred to as a “continuous nanostructure”, or between several hundreds of picometers to several hundreds of nanometers, in which the nanostructure is referred to as a “discontinuous nanostructure”. Thus, the nanostructure of the present embodiments can comprise a nanoparticle, an arrangement of nanoparticles, or any arrangement of one or more nanoparticles and one or more molecules.

The liquid of the above described composition is preferably an aquatic liquid e.g., water.

According to one preferred embodiment of this aspect of the present invention the nanostructures of the cryoprotective composition of the present invention comprise a core material of a nanometer size enveloped by ordered fluid molecules, which are in a steady physical state with each other.

Examples of core materials include, without being limited to, a ferroelectric material, a ferromagnetic material and a piezoelectric material.

A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. Preferably, the nanostructures retains the ferroelectric or ferromagnetic properties of the core material, thereby incorporating a particular feature in which macro scale physical properties are brought into a nanoscale environment.

The core material may also have a crystalline structure.

As used herein, the phrase “ordered fluid molecules” refers to an organized arrangement of fluid molecules which are interrelated, e.g., having correlations thereamongst. For example, instantaneous displacement of one fluid molecule can be correlated with instantaneous displacement of one or more other fluid molecules enveloping the core material.

As used herein, the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

Preferably, the ordered fluid molecules of the envelope are identical to the liquid molecules of the cryoprotective composition. The fluid molecules of the envelope may comprise an additional fluid which is not identical to the liquid molecules of the cryoprotective composition and as such the envelope may comprise a heterogeneous fluid composition.

Due to the formation of the envelope of ordered fluid molecules, the nanostructures of the present embodiment preferably have a specific gravity which is lower than or equal to a specific gravity of the liquid.

The fluid molecules may be either in a liquid state or in a gaseous state or a mixture of the two.

A preferred concentration of nanostructures is below 10²⁰ nanostructures per liter and more preferably below 10¹⁵ nanostructures per liter. The concentration of nanostructures is preferably selected according to the particular stage or method of cryopreservation as described herein below.

Preferably the nanostructures in the liquid are capable of clustering due to attractive electrostatic forces between them. Preferably, even when the distance between the nanostructures prevents cluster formation (about 0.5-10 μm), the nanostructures are capable of maintaining long range interactions.

The long range interaction of the nanostructures has been demonstrated by the present Inventor (see Example 2 in the Examples section that follows). The composition of the present embodiment was subjected to temperature changes and the effect of the temperature changes on ultrasonic velocity was investigated. As will be appreciated by one of ordinary skill in the art, ultrasonic velocity is related to the interaction between the nanostructures in the composition. As demonstrated in the Examples section that follows, the composition of the present invention is characterized by an enhanced ultrasonic velocity relative to water.

Without being bound to theory, it is believed that the long-range interactions between the nanostructures lends to the unique characteristics of the cryoprotective composition. One such characteristic is that the nanostructures and liquid are able to enhance the cryoprotective properties of other cryoprotective agents such as glycerol, as demonstrated in the Example section that follows. This is beneficial as it enables addition of a lower concentration of glycerol (or an absence of glycerol) so that potential toxic side effects are reduced. Another characteristic is that the nanostructures and liquid may enhance cryoprotective properties by providing a stabilizing environment. For example, it has been shown that the carrier composition is capable of protecting proteins from heat (FIGS. 50A-B and FIG. 51).

The present inventors have shown that the composition of the present invention comprises an enhanced buffering capacity (i.e. greater than a buffering capacity of water (FIGS. 17-25)) which may also affect the cryoprotective properties of the present invention.

As used herein, the phrase “buffering capacity” refers to the composition's ability to maintain a stable pH stable as acids or bases are added.

Production of the nanostructures according to this aspect of the present invention may be carried out using a “top-down” process. The process comprises the following method steps, in which a solid powder (e.g., a mineral, a ceramic powder, a glass powder, a metal powder, or a synthetic polymer) is heated, to a sufficiently high temperature, preferably more than about 700° C. Examples of solid powders which are contemplated include, but are not limited to, BaTiO₃, WO₃ and Ba₂F₉O₁₂.

Examples of solid powders which are contemplated include, but are not limited to, BaTiO₃, WO₃ and Ba₂F₉O₁₂. Surprisingly, the present inventors have shown that hydroxyapatite (HA) may also be heated to produce the liquid composition of the present invention.

Hydroxyapatite is specifically preferred as it is characterized by intoxocicty and is generally FDA approved for human therapy.

It will be appreciated that many hydroxyapatite powders are available from a variety of manufacturers such as from Sigma Aldrich and Clarion Pharmaceuticals (e.g. Catalogue No. 1306-06-5).

As shown in Table 2, liquid compositions based on HA, all comprised enhanced buffering capacities as compared to water.

The heated powder is then immersed in a cold liquid, below its density anomaly temperature, e.g., 3° C. or 2° C. Simultaneously, the cold liquid and the powder are irradiated by electromagnetic RF radiation, preferably above 500 MHz, which may be either continuous wave RF radiation or modulated RF radiation.

Cryoprotective compositions of the present invention may additionally comprise one or more stabilizing agents. As used herein the phrase “stabilizing agent” refers to an agent that increases cellular viability. The stabilizing agents of the cryoprotective compositions of the present invention and their concentrations are selected according to the cell type and cell environment. Stabilizer concentrations are generally used at between about 1 μM to about 1 mM, or preferably at between about 10 μM to about 100 μM.

In one embodiment the stabilizing agent increases cellular viability by removing harmful substances from the culture medium. The stabilizing agent may remove both naturally occurring substances (i.e. those secreted by cells during growth or cell death) and artificially introduced substances from the culture medium. For example, a stabilizer may be a radical scavenger chemical or an anti-oxidant that neutralizes the deleterious effects attributable to the presence of active oxygen species and other free radicals. Such substances are capable of damaging cellular membranes, (both internal and external), such that cryoprotection and recovery of cellular matter is seriously compromised. If these substances are not removed or rendered otherwise ineffective, their effects on viability are cumulative over time, severely limiting practical storage life. Furthermore, as cells die or become stressed, additional harmful substances are released increasing the damage and death of neighboring cells.

Examples of oxygen radical scavengers and anti-oxidants include that may be used in accordance with this aspect of the present invention include but are not limited to reduced glutathione, 1,1,3,3-tetramethylurea, 1,1,3,3-tetramethyl-2-thiourea, sodium thiosulfate, silver thiosulfate, betaine, N,N-dimethylformamide, N-(2-mercaptopropionyl)glycine, .beta.-mercaptoethylamine, selenomethionine, thiourea, propylgallate, dimercaptopropanol, ascorbic acid, cysteine, sodium diethyl dithiocarbomate, spermine, spermidine, ferulic acid, sesamol, resorcinol, propylgallate, MDL-71,897, cadaverine, putrescine, 1,3- and 1,2-diaminopropane, deoxyglucose, uric acid, salicylic acid, 3- and 4-amino-1,2,4-triazol, benzoic acid, hydroxylamine and combinations and derivatives of such agents.

Stabilizing agents which may be useful in the cryoprotection of plant cell may include agents that hinder or substantially prevent ethylene biosynthesis and/or ethylene action. It is well known that plant cells emit toxic ethylene when stressed. Therefore, prevention of either the generation of ethylene or the action of ethylene will further enhance cell viability and cell recovery from the cryoprotection process.

Examples of ethylene biosynthetic inhibitors that can be used in the present invention include, but are not limited to Rhizobitoxin, Methoxylamine Hydrochloric acid, Hydroxylamine Analogs, alpha.-Canaline, DNP (2,4-SDS (sodium lauryl sulfate) dinitrophenol), Triton X-100, Tween 20, Spermine, Spermidine, ACC Analogs, alpha.-Aminoisobutyric Acid, n-Propyl Gallate, Benzoic Acid, Benzoic Acid Derivatives, Ferulic Acid, Salicylic Acid, Salicylic Acid Derivatives, Sesamol, Cadavarine, Hydroquinone, Alar AMO-1618, BHA (butylated hydroxyanisol), Phenylethylamine, Brassinosteroids, P-chloromercuribenzoate, N-ethylmaleimide, Iodoacetate, Cobalt, Chloride and other salts, Bipyridyl Amino (oxyacetic) Mercuric Chloride and other Acid salts, Salicyl alcohol, Salicin, Nickle, Chloride and other salts, Catechol, Pffloroglucinol, 1,2-Diaminopropane, Desferrioxamine Indomethacin 1,3-Diaminopropane

Examples of inhibitors of ethylene action include but are not limited to Silver Salts, Benzylisothiocyanate, 8-Hydroxyquinoline sulfate, 8-Hydroxyquinoline citrate, 2,5-norbornadiene, N-ethoxycarbonyl-2-ethoxy-1,2-dihydroquinoline, Trans-cyclootene, 7-Bromo-5-chloro-8-hydroxyquinoline, Cis-Propenylphosphonic Acid, Diazocyclopentadiene, Methylcyclopropane, 2-Methylcyclopropane, Carboxylic Acid, Methylcyclopropane carboxylate, Cyclooctadiene, Cyclooctodine (Chloromethyl) and Cyclopropane

Silver ions are also potent anti-ethylene agent in various plants and are known to improve the longevity of plant tissues and cell cultures. Examples of silver salts which may be used in accordance with this aspect of the present invention include Silver Thiosulfte, Silver Nitrate, Silver Chloride, Silver Acetate, Silver Phosphate, Citric Acid Tri-Silver Salt, Silver Benzoate, Silver Sulfate, Silver Oxide, Silver Nitrite, Silver Cyanate, Lactic Acid Silver Salt and Silver Salts of Pentafluoropropionate Hexafluorophosphate and Toluenesulfonic Acid.

In another embodiment, the stabilizing agent increases cellular viability by stabilizing the cell membrane e.g. by intercalating into the lipid bilayer (e.g. sterols, phospholipids, glycolipids, glycoproteins) or stabilizing membrane proteins (e.g. divalent cations). Examples of divalent cations that may be used in the cryoprotective composition of the present invention include, but are not limited to CaCl₂, MnCl₂ and MgCl₂. Sodium is less preferred due to its toxicity at any more than trace concentrations. Preferred concentrations range from about 1 mM to about 30 mM, and more preferably from about 5 mM to about 20 mM and still more preferably at about 10 mM or 15 mM. Divalent cations also reduce freezing temperatures and allows for the more rapid passage of cells through freezing points.

In yet another embodiment, the stabilizing agent increases cellular viability by preventing or minimizing heat-shock. Thus the stabilizing agent may be a heat shock protein or may be a heat-shock protein stabilizer (e.g. a divalent cation, as described hereinabove).

The cryoprotective composition of the present invention may further comprise stabilizers such as growth factors, egg yolk, serum (e.g. fetal calf serum) and antibiotic compounds (e.g. tylosin, gentamicin, lincospectin, and/or spectinomycin).

In addition, the cryoprotective composition of the present invention may comprise growth medium or buffer. The type of media or buffer selected is dependent on the cell type being cryoprotected, and examples are well known in the art. Suitable examples of acceptable cell buffers include phosphate based buffers such as PBS and Tris based buffers such as Tris EDTA. An example of a growth medium that may be added to the cryoprotective composition of the present invention is DMEM.

As mentioned hereinabove, the compositions of the present invention are characterized by cryoprotective properties and as such can be used for cryopreserving cellular matter.

Thus, according to another aspect of the present invention there is provided a method of cryopreserving cellular matter comprising: (a) contacting the cellular matter with a composition comprising nanostructures and a liquid; and (b) subjecting the cellular matter to a cryopreserving temperature.

As used herein, the term “cryopreserving” refers to maintaining or preserving the viability of cellular matter by storing at very low temperatures. Typically, cryopreserving is effected in the presence of a cryoprotective agent. Preferably cellular matter may be cryopreserved for at least five years following the teachings of the present invention.

As used herein, the phrase “cellular matter” refers to a biological material that comprises cells.

Examples of cellular matter which may be cryopreserved in accordance with this aspect of the present invention include prokaryotic and eukaryotic cellular matter (e.g., mammalian, plant, yeast), but are not limited to, a cellular body fluid (e.g., spinal fluid, blood, amniotic fluid, saliva, synovial fluid, vaginal secretions and semen), isolated cells, a cell culture (e.g., cell-line, primary cell culture, yeast or bacteria culture), a cell suspension, immobilized cells, (e.g. scaffold associated), a tissue, an organ or an organism.

Examples of plant cellular matter include but are not limited to growth needles, leaves, roots, barks, stems, rhizomes, callus cells, protoplasts, cell suspensions, organs, meristems, seeds and embryos, as well as portions thereof.

In a particular embodiment, the cellular matter may comprise stem cells, sperms cells or eggs (i.e. oocytes).

In another particular embodiment, the cellular matter may be naïve or genetically modified.

Cellular matter may be obtained from a living organism or cadaver. For example it may be obtained by surgery (e.g., biopsy) or in an ejaculate. Alternatively, cellular matter may be obtained from a laboratory cell culture.

The following summarizes typical cryopreservation procedures for exemplary cellular matter.

Semen

Semen may be obtained from normal, oligospermic, teratospermic or asthenozoospermic males preferably by donation, although it may also be obtained by surgical methods. The sperm is typically subjected to functional tests in order to determine the quantity of sample that is required to be cryopreserved if there is to be a realistic chance of fertilizatation following recovery. Semen samples are typically mixed in a 1:1 ratio with the cryoprotecting composition of the present invention, and frozen in 0.5 ml aliquots in straws using static vapour phase cooling.

Embryo

Embryos are typically cryopreserved at the pre-implantation stage (e.g. blastocyst stage) following in-vitro fertilization. Embryos are selected according to a range of criteria in order to optimize successful cryopreservation (e.g. 1. blastocyst growth rate—growth rate at day 5 should be greater than growth rate at day 6, which in turn should be greater than the growth rate at day 7; 2. overall cell number—number should be greater or equal to 60 cells (depending on the day of development); 3. relative cell allocation to trophectoderm: inner cell mass; 4. blastomere regularity; 5. mononucleation and; 6. DNA fragmentation).

Standard embryo cryopreservation techniques may involve exposing the embryo to the cryoprotecting composition of the present invention diluted in a simple sodium-based salt solution for 5-15 minutes to allow uptake. The embryos may then cooled quickly (−2° C./min) to about 7° C. at which point they may be seeded, cooled slowly (−0.3° C. to −0.5° C./min) to about −30° C. or below, and then plunged directly into liquid nitrogen. A programmable freezer is typically required for controlled rate cooling. The embryos may be thawed using a rapid approach. Embryos can also be rapidly frozen or vitrified, but only using very elevated cryopreservative concentrations (2M to 6M) that are toxic to cells when they are exposed for more than a few minutes.

Oocytes

Preferably, oocytes that are used for cryopreservation are mature. Mature oocytes may be removed by surgical procedures. Oocyte stimulation prior to removal may also be required. Typically oocytes are selected for cryopreservation based on the following criteria; translucence, shape and extrusion of the first polar body. Typical protocols for the cryopreservation of oocytes are described in U.S. Pat. No. 6,500,608 and U.S. Pat. No. 5,985,538.

Stem Cells

Preservation of pluripotent stem cells poses additional challenges to cryobiology since not only must the cells remain viable, but they must also retain their differentiative capacity (i.e., be maintained in an undifferentiated state). Thus, certain signal transduction pathways must remain in place, and the stresses associated with freezing and drying must not induce premature or erroneous differentiation. Stabilizers may be included which maintain the differentiationless phenotype of the cells immediately following thawing.

Typically stem cell cryopreservation protocols include (1) conventional slow-cooling protocols applied to adherent stem cell colonies and (2) vitrification protocols for both adherent stem cell colonies and freely suspended stem cell clumps.

Skin

Skin is typically removed from cadavers or healthy individuals. Animal skin tissue may also be cryopreserved for use in grafting. The skin is typically tissue-typed prior to cryopreservation or following thawing. Skin cells may be cultured and expanded in vitro prior to cryopreservation. Cryopreservation typically requires a fast thaw protocol. The success or failure of the protocol is measured either by graft take to a wound bed or by a cell viability assay.

Ovarian Tissue

Ovarian tissue (whole ovary or a portion thereof) may be removed from healthy or non-healthy women. Examples of diseases in which it may be advantageous to cryopreserve ovarian tissue include cancer, malignant diseases such as thalassemia and certain auto-immune conditions. Healthy women who have a history of early menopause may also desire ovarian tissue cryoproeservation. Following removal or thawing, the tissue may be screened for malignant cells, and assessed for safety for subsequent auto-grafting.

The cellular matter may be conditioned to facilitate the cryoprotection procedure or may be contacted directly with the compositions of the present invention. As used herein the term “conditioning” refers to protecting the cellular matter from the toxic effects of nanostructures and/or cryoprotecting agents and/or the toxic effects of a decreased temperature. For example the cellular matter may be conditioned with stabilizers and subsequently incubated in the presence of the compositions of the present invention. Alternatively, the compositions of the present invention may be initially applied to the cells followed by the addition of stabilizers or other cryoprotective agents.

Examples of stabilizers are described hereinabove.

Additionally or alternatively, the cellular matter may be cold acclimatized prior to cryoprotecting. This may be affected simultaneously or following conditioning with stabilizers and either prior to or simultaneously with incubating with the compositions of the present invention. This prepares cells for the cryopreservation process by significantly retarding cellular metabolism and reducing the shock of rapid temperature transitions through some of the more critical temperature changes. Critical temperature ranges are those ranges at which there is the highest risk of cell damage, for example, around the critical temperatures of ice crystal formation. As known to those of ordinary skill in the art, these temperatures vary somewhat depending upon the composition of the solution. (For water, the principal component of most cell culture mediums, ice crystal formation and reformation occur at about 0° C. to about −50° C.).

Acclimation results in the accumulation of endogenous solutes that decreases the extent of cell dehydration at any given osmotic potential, and contributes to the stabilization of proteins and membranes during extreme dehydration.

Acclimation may be carried out in a stepwise fashion or gradually. Steps may be in decreasing increments of about 0.5° C. to about 10° C. for a period of time sufficient to allow the cells acclimate to the lower temperature without causing damage. The temperature gradient, whether gradual or stepwise, is scaled to have cells pass through freezing points as quickly as possible. Preferably, acclimation temperatures are between about 1° C. to about 15° C., more preferably between about 2° C. to about 10° C. and even more preferably about 4° C. Cells may be gradually, in a step-wise or continuous manner, or rapidly acclimated to the reduced temperature. Techniques for acclimation are well known to those of ordinary skill and include commercially available acclimators. Gradual acclimation comprises reducing incubation temperatures about 1° C. per hour until the target temperature is achieved. Gradual acclimation is most useful for those cells considered to be most sensitive and difficult to cryoprotect. Stepwise acclimation comprises placing the cells in a reduced temperature for a period of time, a subsequently placing in a further reduced temperature for another period of time. These steps may be repeated as required.

Lyophilization of cellular matter may also be performed prior to cryoprotection. Lyophilization is directed to reducing the water content of the cells by vacuum evaporation. Vacuum evaporation involves placing the cells in an environment with reduced air pressure. Depending on the rate of water removal desired, the reduced ambient pressure operating at temperatures of between about −30° C. to −50° C. may be at 100 torr, 1 torr, 0.01 torr or less. Under conditions of reduced pressure, the rate of water evaporation is increased such that up to 65% of the water in a cell can be removed overnight. With optimal conditions, water removal can be accomplished in a few hours or less. Heat loss during evaporation maintains the cells in a chilled state. By careful adjustment of the vacuum level, the cells may be maintained at a cold acclimation temperature during the vacuum evaporation process. A strong vacuum, while allowing rapid water removal exposes the cells to the danger of freezing.

Freezing may be controlled by applying heat to the cells directly or by adjustment of the vacuum level. When the cells are initially placed in the evaporative chamber, a high vacuum may be applied because the residue heat in the cells will prevent freezing. As dehydration proceeds and the cell temperature drops, the vacuum may be decreased or heating may be applied to prevent freezing. The semi-dry cells may have a tendency to scatter in an evaporative chamber. This tendency is especially high at the end of the treatment when an airstream is allowed back into the chamber. If the air stream proximates the semi-dry cells, it may cause the cells to become airborne and cause cross contamination of the samples. To prevent such disruptions, evaporative cooling may be performed in a vacuum centrifuge wherein the cells are confined to a tube by centrifugal force while drying. The amount of water removed in the process may be monitored periodically by taking dry weight measurement of the cells.

Heat shock treatment may also be performed as an alternative to acclimation prior to cryoprotection. Heat-shock treatment is known to induce de novo synthesis of certain proteins (heat-shock proteins) that are supposed to be involved in adaptation to stress. In addition, heat-shock treatment acts to stabilize membranes and proteins. It tends to improve the survival of cells following cryopreservation by about 20% to about 40%. This procedure involves the incubation of cellular matter (either conditioned or not) in a water-bath shaker at between about 31° C. to about 45° C. preferably between about 33° C. to about 40° C. and more preferably at about 37° C. Culturing is performed from a few minutes to a few hours, preferably from about one hour to about six hours, and more preferably from about two hours to about four hours.

As mentioned hereinabove, the method of this aspect of the present invention is effected by contacting (incubating) the cellular matter with the compositions of the present invention. Preferably, the contacting acts to equilibrate intracellular and/or extracellular concentrations of the nanostructures. The composition of this aspect of the present invention may be added directly to the cellular matter or may be diluted into the medium where the cellular matter is being incubated. To minimize the time required for equilibration, contacting may be performed at about room temperature, although optimal temperature and other conditions for loading will preferably match conditions such as medium, light intensities and oxygen levels that maintain a cell viable.

The compositions of the present invention may be applied directly to the cellular matter or may be diluted in cellular matter incubating mediums, such as culture mediums. Additionally a stepwise incubation (contacting) may be effected. Thus for example, stepwise contacting can be effected such that the cellular matter is incubated in the presence of an increasing concentration of nanostructures. Thus, for example, the cellular matter may be initially contacted with a composition comprising 1010 nanostructures per liter and finally contacted with a composition comprising 1015 nanostructures per liter.

Stepwise contacting is sometimes desired to facilitate delivery of the nanostructures to cells as it is somewhat gentler than single dose loading. Time increments or interval between additions for stepwise loading may range from minutes to hours or more, but are preferable from about one to about ten minutes, more preferably from about one to about five minutes and still more preferably about one or about two minute intervals. The numbers of additions in a stepwise contacting procedure is typically whatever is practical and can range from very few to a large plurality. Preferably, there are less than about twenty additions, more preferably less than about ten and even more preferably about five. Interval periods and numbers of intervals are easily determined by one of ordinary skill in the art for a particular type of cell and loading agent. Incubation times range from minutes to hours as practical.

The cryoprotecting agents or nanostructures in the composition of the present invention may be at a high enough concentration, such that contacting triggers vitrification of the cellular matter.

Vitrification procedures involve gradual or stepwise osmotic dehydration of the cellular matter by direct exposure to concentrated solutions prior to quenching in liquid nitrogen.

Prior to vitrifying, the cellular matter may be incubated with the compositions of the present invention wherein their concentration is not high enough to bring about vitrification. This primarily serves to prevent dehydration-induced destabilization of cellular membranes and possibly proteins. These compositions may optionally be removed prior to vitrification. If the composition remains, the concentration of nanostructures may be increased either gradually or in a stepwise fashion to facilitate vitrification. Other cryoprotecting agents apart from those used to initially contact the cellular matter may be added, or alternatively the identical agents may be added, but at higher concentrations, also in a step-wise or gradual fashion as discussed hereinabove. Concentrations of cryoprotecting agents may range from about 4 M to about 10 M, or between about 25% to about 60%, by weight. This produces an extreme dehydration of the sample cells. Solutions in excess of 7 M typically remove more than 90% of the osmotically active water from the cells; however, precise concentrations for each agent can be empirically determined. Cryoprotecting agents which may be used for vitrification include DMSO, propylene glycol, mannitol, glycerol, polyethylene glycol, ethylene glycol, butanediol, formamide, propanediol and mixtures of these substances.

To minimize the injurious consequences of exposure to high concentrations of cryoprotecting agents or nanostructures, dehydration may be performed at about 0° C. to about 4° C. with the time of exposure as brief as possible. Under these conditions, there is no appreciable influx of additional cryoprotecting agents into the cellular matter because of the difference in the permeability coefficient for water and solutes. As a result, the cellular matter remains contracted and the increase in cytosolic concentration required for vitrification is attained by dehydration.

Cellular matter which has been contacted with compositions of the present invention is cryopreserved by freezing to cryopreservation temperatures. The rate of freezing must strike a balance between the damage caused to cells by mechanical forces during quick freezing and the damage caused to cells by osmotic forces during slow freezing. Different optimal cooling rates have been described for different cells. It has been suggested that the different optimal cooling rates are due to the differences in cellular ice nucleation constants and in phase transition temperature of the cell membrane for different cell types (PCT Publication No. WO 98/14058; Karlsson et al., Biophysical J 65: 2524-2536, 1993). Freezing rates between −1° C. per minute and −10° C. per minute are preferred in the art (Karlsson et al., Biophysical J 65: 2524-2536, 1993). Freezing should be sufficiently rapid to inhibit ice crystal formation. The freezing time should be around 5 minutes or 4 minutes, 3 minutes, 2 minutes, or one minute or less. The critical freezing time should be measured from the frame of reference of a single cell. For example, it may take 10 minutes to pour a large sample of cells into liquid nitrogen, however the individual cell is frozen rapidly by this method.

As mentioned above, the cellular matter may be vitrified. Under those conditions, the cellular matter may be cooled at extremely rapid rates (supercooling) without undergoing intercellular or intracellular ice formation. As well as obviating all of the factors that affect ice formation, rapid cooling also circumvents problems of chilling sensitivity of some cellular matter.

Cellular matter may be directly frozen. Direct freezing methods include dripping, spraying, injecting or pouring cells directly into a cryogenic temperature fluid such as liquid nitrogen or liquid helium. Cellular matter may also be directly contacted to a chilled solid, such as a liquid nitrogen frozen steel block. The cryogenic temperature fluid may also be poured directly onto the cellular matter. The direct method also encompasses contact cells with gases, including air, at a cryogenic temperature. A cryogenic gas stream of nitrogen or helium may be blown directly over or bubbled into a cell suspension. Indirect method involved placing the cells in a container and contacting the container with a solid, liquid, or gas at cryogenic temperature. Examples of containers include plastic vials, glass vials, ampules which are designed to withstand cryogenic temperatures. The container for the indirect freezing method does not have to be impermeable to air or liquid. For example, a plastic bag or aluminum foil is adequate. Furthermore, the container may not necessarily be able to withstand cryogenic temperatures. A plastic vial which cracks but remain substantially intact under cryogenic temperatures may also be used. Cells may also be frozen by placing a sample of cells on one side of a metal foil while contacting the other side of the foil with a gas, solid, or liquid at cryogenic temperature.

Compositions of the present invention may be included in containers suitable for cryopreservation. The container is preferably impervious to the chemicals which it is designed to withhold—for example nanostructures and additional cryoprotecting agents as discussed herein below. The container is preferably made of a material that can withstand cryogenic temperatures. Preferably the container is flexible so that it can absorb volume changes of the various components during the freeze/thaw cycles. Even more preferably, the container of this aspect of the present invention comprises an open tube.

Cryopreserved cellular matter may be maintained at temperatures appropriate for cryo-storage. Final storage temperature is dependent on cell type, but is generally known in the art to be approximately −80° C. to −196° C., the temperatures maintained by dry ice and liquid nitrogen freezers, respectively. Preferably, cells are maintained in liquid nitrogen (about −196° C.), liquid argon, liquid helium or liquid hydrogen. These temperatures will be most appropriate for long term storage of cells, and further, temperature variations can be minimized. Long term storage may be for months and preferably for many years without significant loss of cell viability upon recovery. Short term storage, storage for less than a few months, may also be desired wherein storage temperatures of −150° C., −100° C. or even −50° C. may be used. Dry ice (carbon dioxide) and commercial freezers may be used to maintain such temperatures.

Suitable thawing and recovery is essential to cell survival and to recovery of cells in a condition substantially the same as the condition in which they were originally frozen. As the temperature of the cryoprotected cellular matter is increased during thawing, small ice crystals consolidate and increase in size. Large intracellular ice crystals are generally detrimental to cell survival. To prevent this from occurring, cryoprotected cellular matter should be thawed as rapidly as possible. The rate of heating may be at least about 30° C. per minute to 60° C. per minute. More rapid heating rates of 90° C. per minute, 140° C. per minute to 200° C. or more per minute can also be used. While rapid heating is desired, most cells have a reduced ability to survive incubation temperature significantly above room temperature. To prevent overheating, the cell temperature is preferably monitored. Any heating method can be employed including conduction, convection, radiation, electromagnetic radiations or combinations thereof. Conduction methods involve immersion in water baths, placement in heat blocks or direct placement in open flame. Convection methods involve the use of a heat gun or an oven. Radiation methods involve, for example, heat lamps or ovens such as convection or radiation ovens. Electromagnetic radiation involves the use of microwave ovens and similar devices. Some devices may heat by a combination of methods. For example, an oven heats by convection and by radiation. Heating is preferably terminated as soon as the cells and the surrounding solutions are in liquid form, which should be above 0° C. Since the cryoprotected cellular matter is frozen in the presence of nanostructures and possibly other agents that depress the freezing point, the frozen cells may liquify at a temperature below 0° C. such as at about −10° C.-20° C.-30° C. or −40° C. Thawing of the cryoprotected cells may be terminated at any of these temperatures or at a temperature above 0° C.

Dilution of the composition comprising nanostructures and liquid and its subsequent removal is typically performed as rapidly as possible and as soon as possible following thawing of the cryoprotected cellular matter. If there is a high concentration of nanostructures or cryoprotecting agent in the composition, it is preferred to effect the dilution of the suspending medium while minimizing osmotic expansion. Therefore, dilution of the suspending medium and efflux of the nanostructures or other cryoprotecting agent from within the cellular matter may be accomplished by dilution in a hypertonic medium or a step-wise dilution.

Thawed cells can be gradually acclimated to conditions that allow cells to function normally or if the cellular matter is to be grown following thawing conditions that encourage growth. Cryoprotecting agents may be cytotoxic, cytostatic or mutagenic, and are preferably removed from the thawed cellular matter at a rate which would not harm the cells. A number of removal methods may be used such as resuspension and centrifugation, dialysis, serial washing, bioremediation and neutralization with chemicals, or electromagnetic radiation. The rapid removal of nanostructures and other cryoprotecting agents may increase cell stress and death and thus the removal step may have to be gradual. Removal rates may be controlled by serial washing with solutions that contain less nanostructures or cryoprotecting agents.

Thawing and post-thaw treatments may be performed in the presence of stabilizers (as described hereinabove) to ensure survival and minimize genetic and cellular damage. The stabilizers such as, for example, divalent cations or ethylene inhibitors, reduce, eliminate or neutralize damaging agents which results from cryopreservation. Such damaging agents include free radicals, oxidizers and ethylene.

Preferably, the cellular matter comprises fully-functioning cells so as to increase the percentage of cells that survive following thawing. As described in the Examples section which follows, abnormal sperm cells which had a low pregnancy potential, had a decreased survival rate following freezing stress in the presence of the cryoprotective composition of the present invention than normal sperm cells. Thus, cryoprotecting a mixture of functioning and non-functioning sperm cellular matter in compositions of the present invention may increase the ratio of functioning: non-functioning cells, thereby improving chances of fertilization following thawing.

Preferably at least 10% of the cells in the cellular matter are fully functioning and viable (e.g. sperm cells should be motile, capable of fertilizing an oocyte and should not comprise fragmented DNA) and more preferably 20%, more preferably 30%, more preferably 40%, more preferably 50%, more preferably 60%, more preferably 70%, more preferably 80%, and even more preferably 90%.

After thawing, the cellular matter may optionally be assayed for viability or may be used immediately for transplantation. Viability may be determined by histological and functional methods. Cells are assayed by histological methods known in the art, including, for example, morphological index, exclusion of vital stains, and intracellular pH.

One or more in vitro assays are preferably used to establish functionality of cellular matter. Assays or diagnostic tests well known in the art can be used for these purposes. See, e.g., METHODS IN ENZYMOLOGY, (Abelson, Ed.), Academic Press, 1993. For example, an ELISA (enzyme-linked immunosorbent assay), chromatographic or enzymatic assay, or bioassay specific for the secreted product can be used.

Specifically, if the cellular matter contains sperm, its condition may be analyzed by wave motion analysis, motility assays, and viability counts. For example, a gross microscopic analysis of the semen can be conducted by analyzing wave motion under low magnification (e.g. 10 fold) and ascribing a score for motion from 0-5, with 0 being no wave motion and 5 being rapid wave motion with eddies. Secondly, under higher magnification (e.g. 40 fold) the number of motile sperm can be counted and scored as a percentage of total sperm. This percentage is later multiplied by the concentration/count to determine the number of visibly viable sperm. Sperm concentration can be determined by various procedures: a microcuvette containing semen diluted 1:10 with 0.9% saline is assayed in a Spermacue photometer; or a series of dilutions (1:1000) of the sperm are made and counted with a hemocytometer.

The percentage of viable sperm ratio can be determined by placing a 15 μl drop of extended sample of sperm on a microscope slide with a 15 lilldrop of a Live/Dead stain (Morphology Stain, Lane Manufacturing, Inc., Denver Colo.). A thin smear is prepared after mixing the two drops. The sample is air dried, and then 200 individual sperm are counted by staining with the vital dye under the microscope with a 100 fold oil immersion lens.

Lastly, a sperm's integrity can be assayed by observation of the sperm's acrosomal cap and tail morphology using the Spermac stain. Another microscope slide is prepared with a 15 μl drop of sperm, air dried, and then stained with Spermac following the manufacturer's specification. The overall quality and morphology of the sample is determined by scoring acrosomal caps as intact or non-intact and by counting the number normal tails per 200 individual sperm.

As used herein, the term “about” means ±20%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S., Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### The Effect of Diluted Liquid Comprising Nanostructures with Standard Cryoprotective Solution on Sperm Quality Postfreezing and Thawing

In order to ascertain whether the addition of liquid comprising nanostructures to a standard cryoprotective buffer improves its cryoprotection capabilities, sperm samples were frozen either in the presence or absence of the liquid comprising nanostructures and sperm characteristics were analyzed following thawing.

Materials and Methods

Measurement of sperm motility: Sperm motility was measured under a light microscope, with the aid of a Helber small camera, by counting the number of motile sperm cells.

Measurement of sperm viability: Sperm viability was measured by Eosine Nigrozine staining.

Measurement of sperm DNA fragmentation: Sperm DNA fragmentation was measured by SCSA (Sperm Chromatin Structural Assay).

Measurement of sperm ability to fertilize an egg: The ability of sperm to fertilize an egg was measured by MSOM (motile sperm organelle morphology examination). This examines the number of sperm cells with specific normal morphology and progressive motility, each shown in the literature to act as a marker for fertile cells.

Materials: The standard cryoprotective buffer (TES buffer) comprising TRIS buffer, egg yolk and glycerol was obtained from Irvine scientific (Santa Anna, Calif.).

Experimental procedure: Sperm samples were donated by sub-fertile males (males attending a male fertility clinic) and frozen in the PLANER KRYO-10 instrument using a gradual temperature reducing programme. The specimens were frozen either in the presence of TES (50% semen, 50% TES) or the novel cryoprotective buffer (50% semen, 25% TES and 25% Neowater™ (Do Coop technologies, Israel). The frozen semen was thawed after two days for analysis. The protective effects of the two buffers following freezing on semen quality were compared with a non-frozen native sample of the same semen. The experiment was repeated three times.

Results and Conclusion

The results are summarized in table 1 herein below.

TABLE 1

| | | | liquid comprising |
|---|---|---|---|
| | | Standard | nanostructures |
| | Native | cryopreservation | cryoprotection |
| Motility (%) | 34.5 ± 0.7 | 3.7 ± 0.4 | 5.6 ± 1.6 |
| Viability (%) | 76.5 ± 4.9 | 47.0 ± 2.3 | 49.5 ± 4.9 |
| Normal cells (%) | 1.8 ± 1.4 | 2.5 ± 3.5 | 6.8 ± 2.5 |
| Sperm DNA | 5.4 ± 3.4 | 14.6 ± 1.6 | 12.0 ± 0.7 |
| fragmentation (%) | | | |

Values are the mean ± standard deviation, n = 3

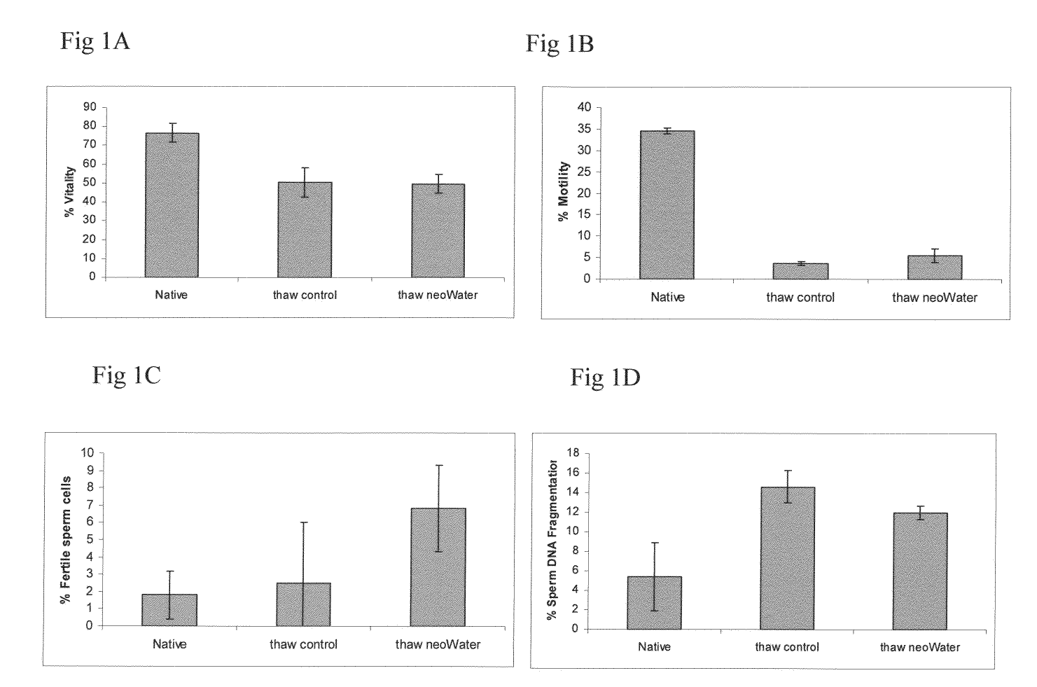

As can be seen from table 1 hereinabove and as depicted in FIGS. 1A-D when the freezing cocktail contains the liquid comprising nanostructures there is an improvement in sperm motility, viability and DNA fragmentation, with a higher percentage of normal cells surviving.

Thus, it can be concluded that abnormal sperm cells which have low pregnancy potential do not survive freezing stress in the presence of the liquid comprising nanostructures.

### Example 2

### Ultrasonic tests

The composition of the present invention has been subjected to a series of ultrasonic tests in an ultrasonic resonator.

Methods

Measurements of ultrasonic velocities in the composition of the present invention (referred to in the present Example as Neowater™) and double distilled (dist.) water were performed using a ResoScan® research system (Heidelberg, Germany).

Calibration: Both cells of the ResoScan® research system were filled with standard water (demin. Water Roth. Art.3175.2 Charge:03569036) supplemented with 0.005% Tween 20 and measured during an isothermal measurement at 20° C. The difference in ultrasonic velocity between both cells was used as the zero value in the isothermal measurements and temperatures scans as further detailed hereinbelow.

Isothermal Measurements: Cell 1 of the ResoScan® research system was used as reference and was filled with dist. Water (Roth Art. 34781 lot#48362077). Cell 2 was filled with the carrier composition of the present invention. Absolute Ultrasonic velocities were measured at 20° C. In order to allow comparison of the experimental values, the ultrasonic velocities were corrected to 20.000° C.

Results

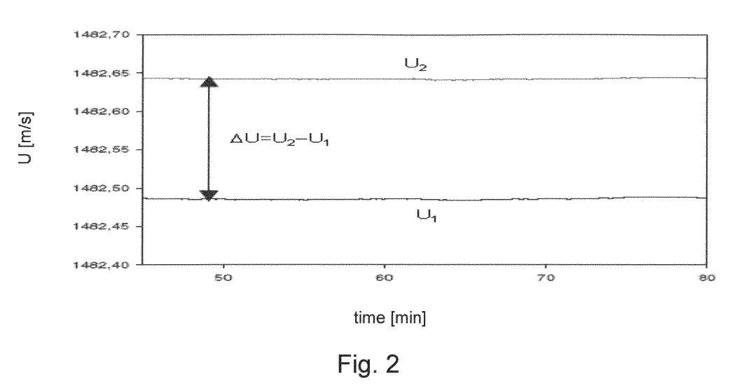

FIG. 2 shows the absolute ultrasonic velocity U as a function of observation time, as measured at 20.051° C. for the carrier composition of the present invention (U₂) and the dist. water (U₁). Both samples displayed stable isothermal velocities in the time window of observation (35 min).

Table 2 below summarizes the measured ultrasonic velocities U₁, U₂ and their correction to 20° C. The correction was calculated using a temperature-velocity correlation of 3 m/s per degree centigrade for the dist. Water.

TABLE 2

| Sample | Temp | U |
|---|---|---|
| dist. water | 20.051° C. | 1482.4851 |
| Neowater ™ | | 1482.6419 |
| dist. water | 20° C. | 1482.6381 |
| Neowater ™ | | 1482.7949 |

As shown in FIG. 2 and Table 2, differences between dist. water and the carrier composition of the present invention were observed by isothermal measurements. The difference ΔU=U₂−U₁ was 15.68 cm/s at a temperature of 20.051° C. and 13.61 cm/s at a temperature of 20° C. The value of ΔU is significantly higher than any noise signal of the ResoScan® system. The results were reproduced once on a second ResoScan® research system.

### Example 3

### Buffering Capacity of the Composition Comprising Nanostructures

The effect of the composition comprising nanostructures on buffering capacity was examined.

Materials and Methods

Phenol red solution (20 mg/25 ml) was prepared. 290 μl was added to 13 ml RO water or various batches of water comprising nanostructures (Neowater™—Do-Coop technologies, Israel). It was noted that each water had a different starting pH, but all of them were acidic, due to their yellow or light orange color after phenol red solution was added. 2.5 ml of each water+phenol red solution were added to a cuvette. Increasing volumes of Sodium hydroxide were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Results

Table 3 summarizes the absorbance at 557 nm of each water solution following sodium hydroxide titration.

TABLE 3

| | | | | | | | μl of 0.02 |
|---|---|---|---|---|---|---|---|
| | NW 2 | | NW 4 | NW 5 | | | M sodium |
| NW 1 | AB 1- | NW 3 | Alex- | HA-99- | NW | | hydroxide |
| HAP | 2-3 | HA 18 | ander | X | 6 | RO | added |
| 0.026 | 0.033 | 0.028 | 0.093 | 0.011 | 0.118 | 0.011 | 0 |
| 0.132 | 0.17 | 0.14 | 0.284 | 0.095 | 0.318 | 0.022 | 4 |
| 0.192 | 0.308 | 0.185 | 0.375 | 0.158 | 0.571 | 0.091 | 6 |
| 0.367 | 0.391 | 0.34 | 0.627 | 0.408 | 0.811 | 0.375 | 8 |
| 0.621 | 0.661 | 0.635 | 1.036 | 0.945 | 1.373 | 0.851 | 10 |
| 1.074 | 1.321 | 1.076 | 1.433 | 1.584 | 1.659 | 1.491 | 12 |

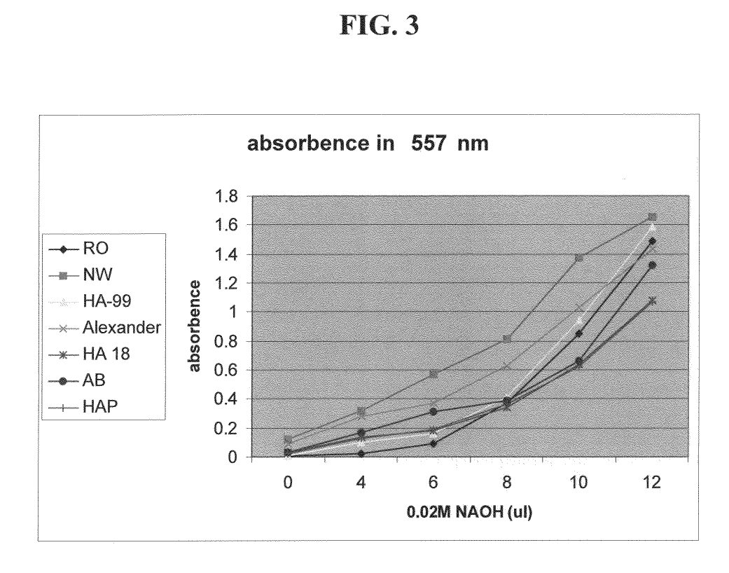

As illustrated in FIG. 3 and Table 2, RO water shows a greater change in pH when adding Sodium hydroxide. It has a slight buffering effect, but when absorbance reaches 0.09 A, the buffering effect “breaks”, and pH change is greater following addition of more Sodium hydroxide. HA-99 water is similar to RO. NW (#150905-106) (Neowater™), AB water Alexander (AB 1-22-1 HA Alexander) has some buffering effect. HAP and HA-18 shows even greater buffering effect than Neowater™.

In summary, from this experiment, all new water types comprising nanostructures tested (HAP, AB 1-2-3, HA-18, Alexander) shows similar characters to Neowater™, except HA-99-X.

### Example 4

### Buffering Capacity of the Liquid Composition Comprising Nanostructures

The effect of the liquid composition comprising nanostructures on buffering capacity was examined.

Materials and Methods

Sodium hydroxide and Hydrochloric acid were added to either 50 ml of RO water or water comprising nanostructures (Neowater™—Do-Coop technologies, Israel) and the pH was measured. The experiment was performed in triplicate. In all, 3 experiments were performed.

Sodium hydroxide titration: −1 μl to 15 μl of 1M Sodium hydroxide was added.

Hydrochloric acid titration: −1 μl to 15 μl of 1M Hydrochloric acid was added.

Results

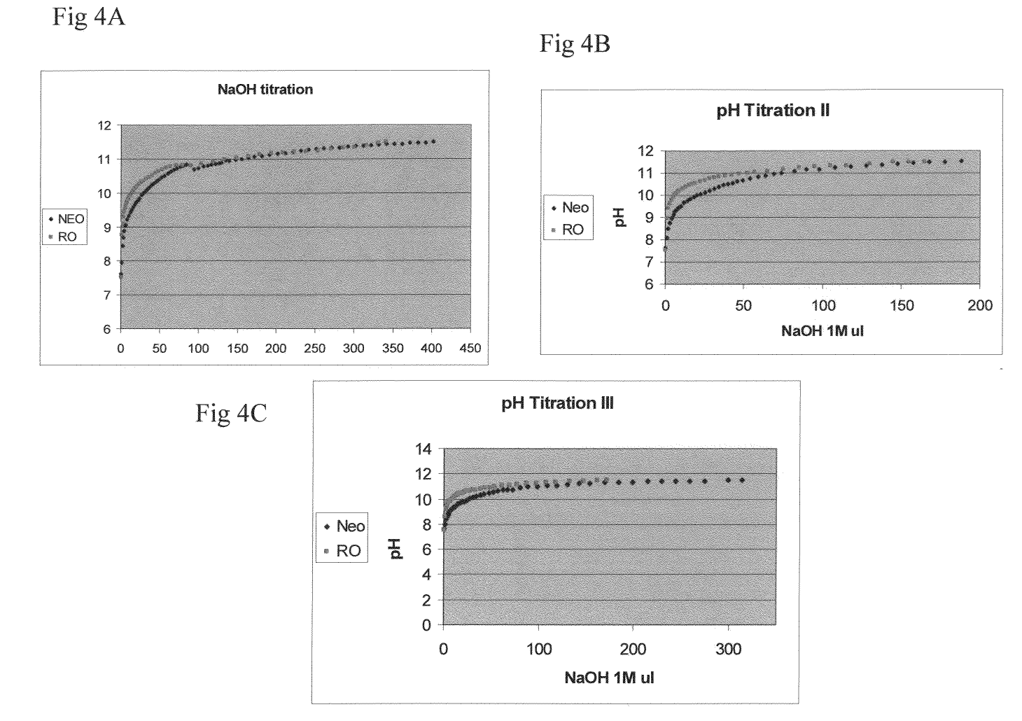

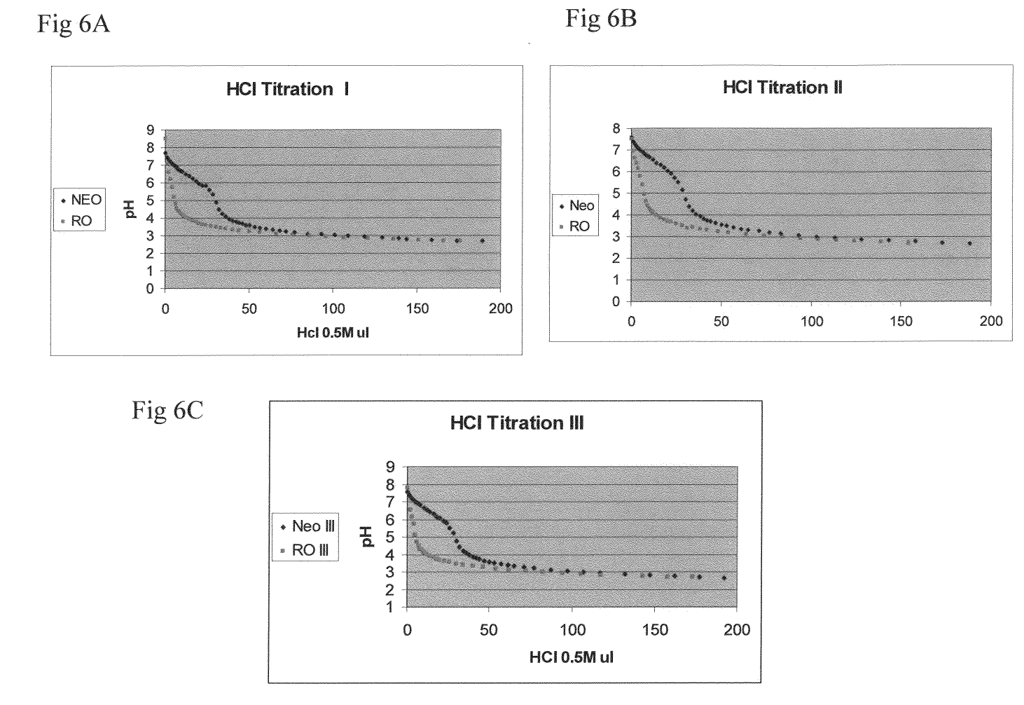

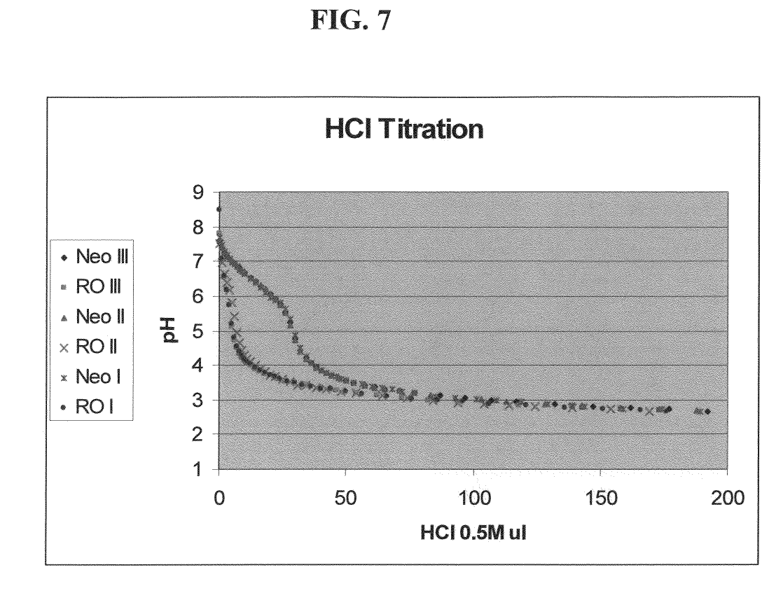

The results for the Sodium hydroxide titration are illustrated in FIGS. 4A-C and 5A-C. The results for the Hydrochloric acid titration are illustrated in FIGS. 6A-C and FIG. 7.

The water comprising nanostructures has buffering capacities since it requires greater amounts of Sodium hydroxide in order to reach the same pH level that is needed for RO water. This characterization is more significant in the pH range of −7.6-10.5. In addition, the water comprising nanostructures requires greater amounts of Hydrochloric acid in order to reach the same pH level that is needed for RO water. This effect is higher in the acidic pH range, than the alkali range. For example: when adding 10 μl Sodium hydroxide 1M (in a total sum) the pH of RO increased from 7.56 to 10.3. The pH of the water comprising nanostructures increased from 7.62 to 9.33. When adding 10 μl Hydrochloric acid 0.5M (in a total sum) the pH of RO decreased from 7.52 to 4.31 The pH of water comprising nanostructures decreased from 7.71 to 6.65. This characterization is more significant in the pH range of −7.7-3.

### Example 5

### Buffering Capacity of the Liquid Composition Comprising Nanostructures

The effect of the liquid composition comprising nanostructures on buffering capacity was examined.

Materials and Methods

Phenol red solution (20 mg/25 ml) was prepared. 1 ml was added to 45 ml RO water or water comprising nanostructures (Neowater™—Do-Coop technologies, Israel). pH was measured and titrated if required. 3 ml of each water+phenol red solution were added to a cuvette. Increasing volumes of Sodium hydroxide or Hydrochloric acid were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Hydrochloric acid Titration:

### RO: 45 ml pH 5.8

1 ml phenol red and 5 μl Sodium hydroxide 1M was added, new pH=7.85 Neowater™ (# 150905-106): 45 ml pH 6.3

1 ml phenol red and 4 μl Sodium hydroxide 1M was added, new pH=7.19

Sodium Hydroxide Titration:

### I. RO: 45 ml pH 5.78

1 ml phenol red, 6 μl Hydrochloric acid 0.25M and 4 μl Sodium hydroxide 0.5M was added, new pH=4.43

### Neowater™ (# 150604-109): 45 ml pH 8.8

1 ml phenol red and 45 μl Hydrochloric acid 0.25M was added, new pH=4.43

### II. RO: 45 ml pH 5.78

1 ml phenol red and 5 μl Sodium hydroxide 0.5M was added, new pH=6.46 Neowater™ (# 120104-107): 45 ml pH 8.68

1 ml phenol red and 5 μl Hydrochloric acid 0.5M was added, new pH=6.91

Results

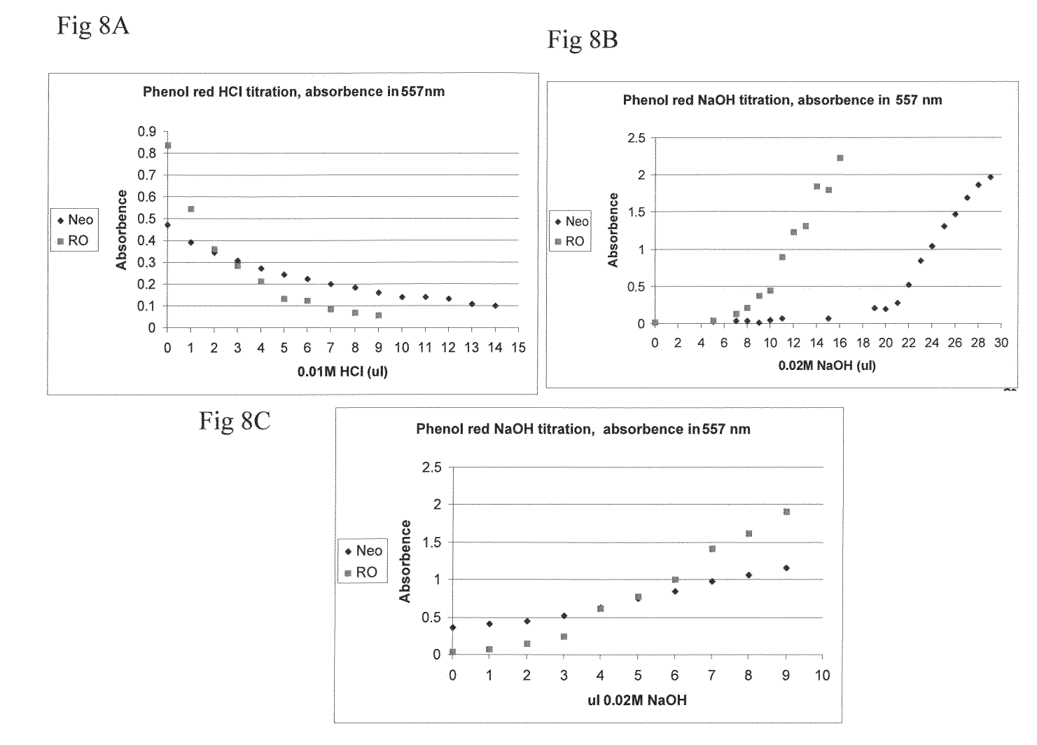

As illustrated in FIGS. 8A-C and 9A-B, the buffering capacity of water comprising nanostructures was higher than the buffering capacity of RO water.

### Example 6

### Buffering Capacity of RF Water

The effect of the RF water on buffering capacity was examined.

Materials and Methods

A few μl drops of Sodium hydroxide 1M were added to raise the pH of 150 ml of RO water (pH=5.8). 50 ml of this water was aliquoted into three bottles.

Three treatments were done:

Bottle 1: no treatment (RO water)

Bottle 2: RO water radiated for 30 minutes with 30 W. The bottle was left to stand on a bench for 10 minutes, before starting the titration (RF water).

Bottle 3: RF water subjected to a second radiation when pH reached 5. After the radiation, the bottle was left to stand on a bench for 10 minutes, before continuing the titration.

Titration was performed by the addition of 1 μl 0.5M Hydrochloric acid to 50 ml water. The titration was finished when the pH value reached below 4.2.

The experiment was performed in triplicates.

Results

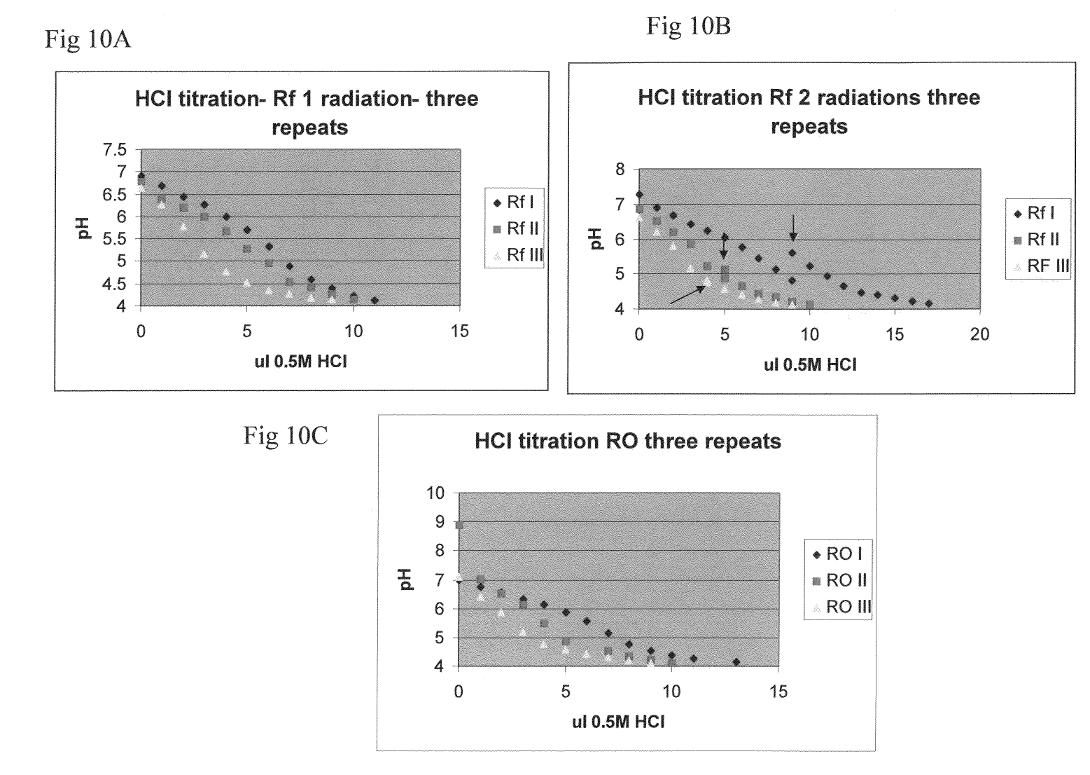

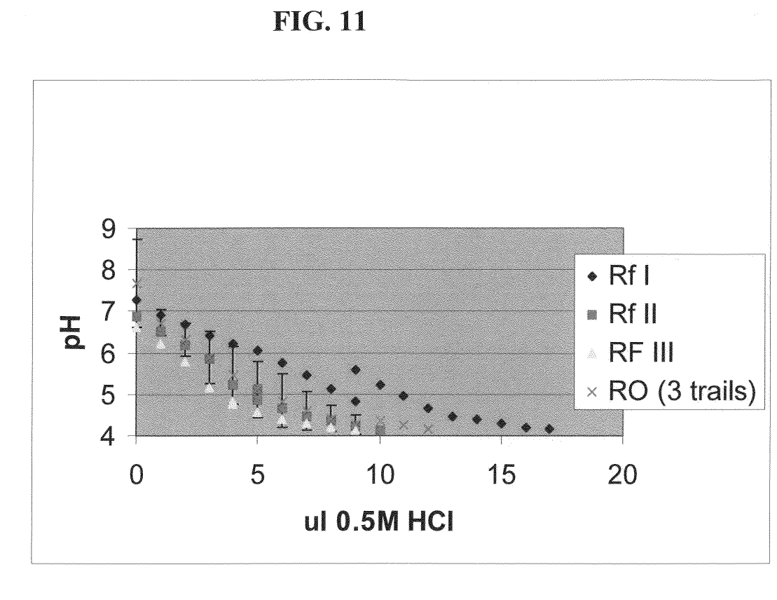

As can be seen from FIGS. 10A-C and FIG. 11, RF water and RF2 water comprise buffering properties similar to those of the carrier composition comprising nanostructures.

### Example 7

### Stabilizing Effect of the Liquid Composition Comprising Nanostructures

The following experiment was performed to ascertain if the liquid composition comprising nanostructures effected the stability of a protein.

Materials and Methods

Two commercial Taq polymerase enzymes (Peq-lab and Bio-lab) were checked in a PCR reaction to determine their activities in ddH₂O(RO) and carrier comprising nanostructures (Neowater™—Do-Coop technologies, Israel). The enzyme was heated to 95° C. for different periods of time, from one hour to 2.5 hours.

2 types of reactions were made:

Water only—only the enzyme and water were boiled.

All inside—all the reaction components were boiled: enzyme, water, buffer, dNTPs, genomic DNA and primers.

Following boiling, any additional reaction component that was required was added to PCR tubes and an ordinary PCR program was set with 30 cycles.

Results

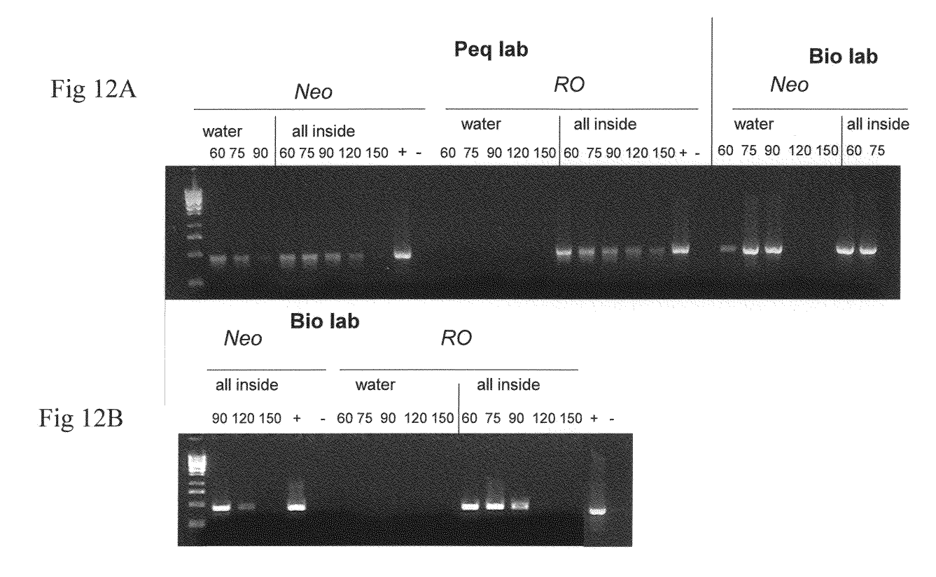

As illustrated in FIGS. 12A-B, the carrier composition comprising nanostructures protected the enzyme from heating, both under conditions where all the components were subjected to heat stress and where only the enzyme was subjected to heat stress. In contrast, RO water only protected the enzyme from heating under conditions where all the components were subjected to heat stress.

### Example 8

### Further Illustration of the Stabilizing Effect of the Carrier Comprising Nanostructures

The following experiment was performed to ascertain if the carrier composition comprising nanostructures effected the stability of two commercial Taq polymerase enzymes (Peq-lab and Bio-lab).

Materials and Methods

The PCR reactions were set up as follows:

Peq-lab samples: 20.4 μl of either the carrier composition comprising nanostructures (Neowater™—Do-Coop technologies, Israel) or distilled water (Reverse Osmosis=RO).

0.1 μl Taq polymerase (Peq-lab, Taq DNA polymerase, 5 U/μl)

Three samples were set up and placed in a PCR machine at a constant temperature of 95° C. Incubation time was: 60, 75 and 90 minutes.

Following boiling of the Taq enzyme the following components were added:

2.5 μl 10× reaction buffer Y (Peq-lab)
0.5 μl dNTPs 10 mM (Bio-lab)
1 μl primer GAPDH mix 10 μmol/μl
0.5 μl genomic DNA 35 μg/μl

Biolab Samples

18.9 μl of either carrier comprising nanostructures (Neowater™—Do-Coop technologies, Israel) or distilled water (Reverse Osmosis=RO).

0.1 μl Taq polymerase (Bio-lab, Taq polymerase, 5 U/μl)

Five samples were set up and placed in a PCR machine at a constant temperature of 95° C. Incubation time was: 60, 75, 90 120 and 150 minutes.

Following boiling of the Taq enzyme the following components were added:

2.5 μl TAQ 10× buffer Mg-free (Bio-lab)

### 1.5 μl MgCl₂ 25 mM (Bio-lab)

0.5 μl dNTPs 10 mM (Bio-lab)
1 μl primer GAPDH mix (10 μmol/μl)
0.5 μl genomic DNA (35 μg/μl)

For each treatment (Neowater or RO) a positive and negative control were made. Positive control was without boiling the enzyme. Negative control was without boiling the enzyme and without DNA in the reaction. A PCR mix was made for the boiled taq assays as well for the control reactions.

Samples were placed in a PCR machine, and run as follows:

PCR Program:

1. 94° C. 2 minutes denaturation

2. 94° C. 30 seconds denaturation

3. 60° C. 30 seconds annealing

4. 72° C. 30 seconds elongation repeat steps 2-4 for 30 times

5. 72° C. 10 minutes elongation

Results

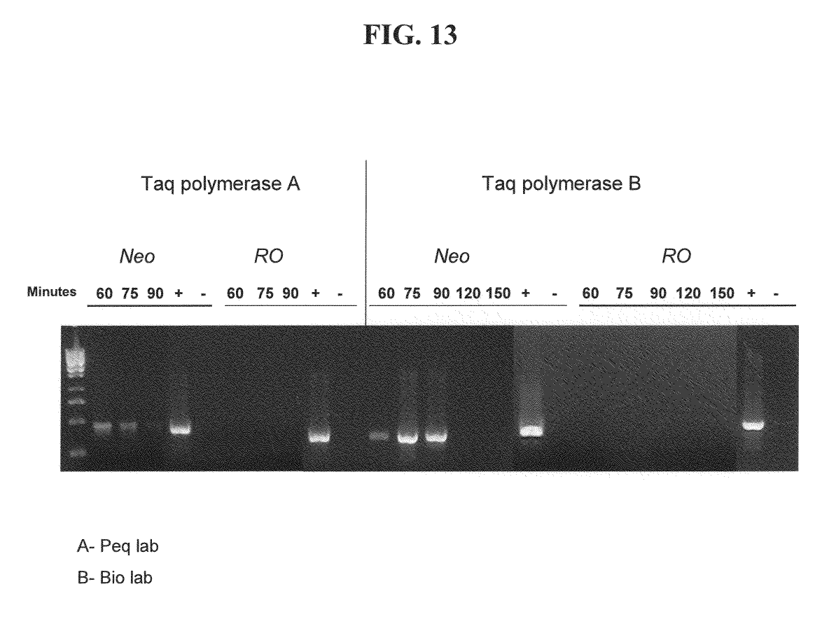

As illustrated in FIG. 13, the liquid composition comprising nanostructures protected both the enzymes from heat stress for up to 1.5 hours.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A cryoprotective composition comprising nanostructures, liquid and at least one cryoprotective agent.

2. A method of cryopreserving cellular matter comprising:
(a) contacting the cellular matter with a composition comprising nanostructures and a liquid; and
(b) subjecting the cellular matter to a cryopreserving temperature, thereby cryopreserving the cellular matter.

3. A method of recovering cryopreserved cellular matter comprising:
(a) contacting cellular matter with a composition comprising nanostructures and a liquid;
(b) subjecting the cellular matter to a cryopreserving temperature to generate cryopreserved cellular matter;
(c) thawing said cryopreserved cellular matter; and
(d) removing said composition; thereby recovering cryopreserved cellular matter.

4. (canceled)

5. A cryopreservation container comprising nanostructures and a liquid.

6. The method of claim 2, wherein the cryoprotective composition further comprises at least one cryoprotective agent.

7-19. (canceled)

20. The cryoprotective composition of claim 1, wherein said nanostructures are formulated from hydroxyapatite.

21. The cryoprotective composition of claim 1, comprising less than 10% by volume glycerol.

22. The cryoprotective composition of claim 1 being devoid of glycerol.

23. (canceled)

24. The cryoprotective composition of claim 1 further comprising a stabilizer.

25-28. (canceled)

29. The method of claim 2, wherein said cellular matter is selected from the group comprising a body fluid, a cell culture, a cell suspension, a cell matrix, a tissue, an organ and an organism.

30. The method of claim 29, wherein said body fluid is semen.

31-35. (canceled)

36. The method of claim 2, wherein said cellular matter is selected from the group consisting of a stem cell, a sperm, an egg and an embryo.

37. (canceled)

38. The method of claim 2, further comprising conditioning the cellular matter prior to step (a).

39. (canceled)

40. The method of claim 2, wherein step (a) and step (b) are performed simultaneously.

41. The method of claim 3 wherein said cryopreserving temperature is less than about −80° C.

42. The cryopreservation container of claim 5, further comprising at least one cryoprotective agent.

43. The method of claim 3, wherein said cellular matter is selected from the group comprising a body fluid, a cell culture, a cell suspension, a cell matrix, a tissue, an organ and an organism.

44. The method of claim 43, wherein said body fluid is semen.

45. The method of claim 3, wherein said cellular matter is selected from the group consisting of a stem cell, a sperm, an egg and an embryo.
